SEQUENCE LISTING (35 sequences)

SEQ ID NO: 1 - protein, 104 aa, Mus musculus
PSPGGLVAPAFTRSSSRKRARPPAEPGSDQPAPLARRRLRLPGLDSCPSSLPEPSSPAEPSPPADPSPPADPGSPVCPSPVKRTKSTTVYVGQQPGKIPSEDSV

SEQ ID NO: 2 - protein, 107 aa, Mus musculus
MNLSMKQKQEGAQENVKNSPVPRRTLKMIQPSADGSLVGRENELPKGLFKRKLWDDQLASQTSSCGPEANENKDVGDLTQEAFDLISKENPSSQYWKEVAEQRRKAL

SEQ ID NO: 3 - protein, 91 aa, Homo sapiens
PSPARPALRAPASATSGSRKRARPPAAPGRDQARPPARRRLRLSVDEVSSPSTPEAPDIPACPSPGQKIKKSTPAAGQPPHLTSAQDQDTI

SEQ ID NO: 4 - protein, 110 aa, Homo sapiens
MNPSMKQKQEEIKENIKNSSVPRRTLKMIQPSASGSLVGRENELSAGLSKRKHRNDHLTSTTSSPGVIVPESSENKNLGGVTQESFDLMIKENPSSQYWKEVAEKRRKAL

SEQ ID NO: 5 - DNA, 8818 bp, Artificial Sequence
  pX330-mG VECTOR DERIVED FROM STREPTOCOCCUS PYOGENES AND MUS MUSCLUS
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggtcttcgagaagacctgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacatgaatctcagtatgaagcagaagcaggagggagcccaagagaatgtgaagaatagtcctgtcccaaggagaacgctgaagatgatccagccttctgcagatggatctcttgttggcagagaaaatgagttgccaaaaggcttgttcaaaaggaagctttgggatgaccagctagcatctcagacttcaagctgtggtccagaagctaatgaaaataaggatgttggagacctcacccaggaagcctttgatcttataagtaaagagaacccatcttctcagtattggaaagaagtggcagagcagcggaggaaagctctcgaattcagccccaagaagaagagaaaggtggaggccagctaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 6 - DNA, 8816 bp, Artificial Sequence
  pX330-mC VECTOR DERIVED FROM STREPTOCOCCUS PYOGENES AND MUS MUSCLUS
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggtcttcgagaagacctgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacccgagccctggtggcctcgtggctcctgcgttcacccggagcagcagccgcaagcgcgcccggcccccagccgaacccgggagtgaccagcccgcgccgctcgcgcgccggaggttacggctgcctggattggactcctgccccagttctctgcctgagcccagttccccagctgagcccagccctccagctgaccctagccctccagctgaccctggctcccccgtttgcccatcccccgtcaagcgaacaaagagtacaactgtttatgttggtcaacagccgggcaagatcccctcagaggactccgtcgaattcagccccaagaagaagagaaaggtggaggccagctaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgcaattccctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 7 - DNA, 28 bp, Mus musculus
tcaagacaaacctgagcacttttcatca

SEQ ID NO: 8 - DNA, 28 bp, Mus musculus
acccaatgtggttaaaggaaagaaccaa

SEQ ID NO: 9 - DNA, 23 bp, Mus musculus
cctgtgttaatctactatgggct

SEQ ID NO: 10 - DNA, 23 bp, Mus musculus
cctagacgggatctatctcactt

SEQ ID NO: 11 - DNA, 20 bp, Mus musculus
tggatctaccccatttctgc

SEQ ID NO: 12 - DNA, 20 bp, Mus musculus
aaattttggtggcattgcat

SEQ ID NO: 13 - DNA, 20 bp, Mus musculus
acagggggacattgcaaata

SEQ ID NO: 14 - DNA, 20 bp, Mus musculus
ccacacaggtaaaggggcta

SEQ ID NO: 15 - DNA, 23 bp, Mus musculus
cctcaatgagtagtttgtttccc

SEQ ID NO: 16 - DNA, 23 bp, Mus musculus
tgtgtccatatagtactaggagg

SEQ ID NO: 17 - DNA, 23 bp, Mus musculus
ccagtctttctagaagatgggcg

SEQ ID NO: 18 - DNA, 30 bp, Mus musculus
ctcagagagcctcggctaggtaggggatcg

SEQ ID NO: 19 - DNA, 30 bp, Artificial Sequence
  SYNTHESIZED ORIGONUCLEOTIDE
acgtcaatggaaagtccctattggcgttac

SEQ ID NO: 20 - DNA, 28 bp, Artificial Sequence
  SYNTHESIZED ORIGONUCLEOTIDE
tgccatgaacaaaggttggctataaaga

SEQ ID NO: 21 - DNA, 28 bp, Mus musculus
gcctgccaagtaactactcttgtgtgct

SEQ ID NO: 22 - DNA, 28 bp, Mus musculus
aatttgatgggggaaaacttgaatgaaa

SEQ ID NO: 23 - DNA, 23 bp, Mus musculus
gtatccaggaacatcttgagtgg

SEQ ID NO: 24 - DNA, 23 bp, Mus musculus
cccggtcaggctctagatagtca

SEQ ID NO: 25 - DNA, 28 bp, Mus musculus
cttgtactcaaaaccttctgccccaact

SEQ ID NO: 26 - DNA, 28 bp, Mus musculus
acttctgtcctctcaggggcactctact

SEQ ID NO: 27 - DNA, 69 bp, Artificial Sequence
  DERIVED FROM BACTERIOPHAGE P1
gtatccaggaacatcttaaacataacttcgtatagcatacattatacgaagttatggcgcgccgagtgg

SEQ ID NO: 28 - DNA, 85 bp, Artificial Sequence
  DERIVED FROM BACTERIOPHAGE P1
cccggtcgcgccaattcgatatcaagctataacttcgtatagcatacattatacgaagttatgcggcccaggctctagatagtca

SEQ ID NO: 29 - protein, 52 aa, Mus musculus
PSPGGLVAPAFTRSSSRKRARPPAEPGSDQPAPLARRRLRLPGLDSCPSSLP

SEQ ID NO: 30 - DNA, 8756 bp, Artificial Sequence
  pX330-pFmC VECTOR DERIVED FROM STREPTOCOCCUS PYOGENES AND MUS MUSCLUS
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggtcttcgagaagacctgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatggactataaggaccacgacggagactacaaggatcatgatattgattacaaagacgatgacgataagatggccatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacccgagccctggtggcctcgtggctcctgcgttcacccggagcagcagccgcaagcgcgcccggcccccagccgaacccgggagtgaccagcccgcgccgctcgcgcgccggaggttacggctgcctggattggactcctgccccagttctctgcctgagcccagttccccagctgagcccagccctccagctgaccctagccctccagctgaccctggctcccccgtttgcccatcccccgtcaagcgaacaaagagtacaactgtttatgttggtcaacagccgggcaagatcccctcagaggactccgtcgaattctaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgcaattccctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 31 - DNA, 8600 bp, Artificial Sequence
  pX330-pCmC VECTOR DERIVED FROM STREPTOCOCCUS PYOGENES AND MUS MUSCLUS
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggtcttcgagaagacctgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatggactataaggaccacgacggagactacaaggatcatgatattgattacaaagacgatgacgataagatggccatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgagcccagttccccagctgagcccagccctccagctgaccctagccctccagctgaccctggctcccccgtttgcccatcccccgtcaagcgaacaaagagtacaactgtttatgttggtcaacagccgggcaagatcccctcagaggactccgtcgaattctaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgcaattccctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 32 - DNA, 8600 bp, Artificial Sequence
  pX330-pMmC VECTOR DERIVED FROM STREPTOCOCCUS PYOGENES AND MUS MUSCLUS
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggtcttcgagaagacctgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatggactataaggaccacgacggagactacaaggatcatgatattgattacaaagacgatgacgataagatggccatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccattacgagaaactgaaagggtctcctgaggataatgaacagaagcaactgtttgtcgagcagcataaacactacttggacgaaataatcgagcagatatcagaattctctaaaagagtgattcttgcagatgccaacctggataaggtgctcagtgcttataataaacaccgcgataagcccatccgcgaacaggctgaaaatattatacacctctttacactcacaaatcttggagcccccgccgcttttaaatactttgataccactatcgatagaaaacggtatacctcaactaaagaggttctcgacgcaacccttatccaccagagcatcacaggactgtacgaaactaggatagatctttcccaattggggggcgacggttcagaccaacctgcaccattggcacgacgacgcttgcgcctcccagggcttgacagttgtccttcaagcttgcctgaaccttcctctccagccgaaccatcccccccagccgaccccagtcctcccgcagaccccggctcacccgtctgcccagaattctaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgcaattccctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 33 - DNA, 8600 bp, Artificial Sequence
  pX330-pNmC VECTOR DERIVED FROM STREPTOCOCCUS PYOGENES AND MUS MUSCLUS
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggtcttcgagaagacctgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatggactataaggaccacgacggagactacaaggatcatgatattgattacaaagacgatgacgataagatggccatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacccgagccctggtggcctcgtggctcctgcgttcacccggagcagcagccgcaagcgcgcccggcccccagccgaacccgggagtgaccagcccgcgccgctcgcgcgccggaggttacggctgcctggattggactcctgccccagttctctgcctgaattctaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgcaattccctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 34 - DNA, 8522 bp, Artificial Sequence
  pX330-pNNmC VECTOR DERIVED FROM STREPTOCOCCUS PYOGENES AND MUS MUSCLUS
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggtcttcgagaagacctgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatggactataaggaccacgacggagactacaaggatcatgatattgattacaaagacgatgacgataagatggccatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacccgagccctggtggcctcgtggctcctgcgttcacccggagcagcagccgcaagcgcgcccggcccccagccgaacccgaattctaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgcaattccctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 35 - DNA, 8522 bp, Artificial Sequence
  pX330-pNCmC VECTOR DERIVED FROM STREPTOCOCCUS PYOGENES AND MUS MUSCLUS
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggtcttcgagaagacctgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatggactataaggaccacgacggagactacaaggatcatgatattgattacaaagacgatgacgataagatggccatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgggagtgaccagcccgcgccgctcgcgcgccggaggttacggctgcctggattggactcctgccccagttctctgcctgaattctaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgcaattccctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

(19) United States
(12) Patent Application Publication
(10) Pub. No.: US 2021/0189404 A1
(43) Pub. Date: Jun. 24, 2021

(54) METHOD FOR PRODUCING FUSION PROTEIN, NUCLEIC ACID, CELL, AND ANIMAL

(71) Applicant: University of Tsukuba, Ibaraki (JP)

(72) Inventors: Seiya MIZUNO, Ibaraki (JP); Fumihiro SUGIYAMA, Ibaraki (JP); Satoru TAKAHASHI, Ibaraki (JP); Saori MIZUNO, Ibaraki (JP)

(21) Appl. No.: 17/057,595

(22) PCT Filed: May 22, 2019
(86) PCT No.: PCT/JP2019/020244

§ 371 Date: Nov. 20, 2020

(30) Foreign Application Priority Data
May 25, 2018 (JP) 2018-101060

(51) Int. Cl. C12N 15/62; A01K 67/027; C07K 14/47; C12N 9/22

(52) U.S. Cl. CPC C12N 15/62; C12N 9/22; C07K 14/47; A01K 67/027

(57) ABSTRACT

A fusion protein including a Cas9 protein and a modifying peptide that modifies the Cas9 protein, in which the modifying peptide includes: a peptide composed of the amino acid sequence described in SEQ ID NO: 29; or a peptide composed of an amino acid sequence in which one or several amino acids are deleted, substituted, or added in the amino acid sequence described in SEQ ID NO: 29 and having activity to localize the Cas9 protein in the nucleus by forming a fusion protein with the Cas9 protein.

### TECHNICAL FIELD

The present invention relates to a method for producing a fusion protein, a nucleic acid, a cell and an animal.

### BACKGROUND ART

With a knock-out (hereafter may be referred to as “KO”) mouse in which a specific gene are disabled, or a knock-in (hereafter may be referred to as “KI”) mouse in which an exogenous gene are introduced into a specific locus, the function of a gene can be directly evaluated in vivo. Hence, such mice are used in many medical and life science studies.

Traditionally, KO or KI mice are created by a gene targeting method using embryonic stem (hereafter may be referred to as “ES”) cells. However, a problem with this method is that production is costly and studies take years. These problems were resolved by genome editing technologies.

In genome editing, a target gene can be knocked out by introducing into a cell an artificial restriction enzyme that recognizes only a specific DNA sequence and cleaves only at that site. It is also possible to knock in an exogenous gene at a target locus by introducing an artificial restriction enzyme into a cell along with donor DNA in which the exogenous gene to be inserted is flanked by neighboring sequences at the cleavage site.

The most powerful artificial restriction enzyme that can be used for genome editing at present is the Clustered Regularly Interspaced Short Palindromic Repeats—CRISPR-Associated Protein 9 (hereinafter may be referred to as “CRISPR-Cas9”) system.

Among CRISPR-Cas9s, Streptococcus pyogenes- (hereinafter may be referred to as “Sp”) derived CRISPR-Cas9 (hereinafter may be referred to as “SpCas9”) is widely used in studies for its ease of use and effectiveness in cleaving DNA.

The Sp-CRISPR-Cas9 system is an RNA-protein complex mainly composed of a single guide RNA (hereinafter may be referred to as “sgRNA”) with the function of recognizing a target sequence and a Cas9 protein with the main function of cleaving the target sequence. A KO mouse can be obtained by simultaneously introducing the sgRNA and Cas9 protein into a fertilized mouse egg.

Further, by simultaneously cleaving upstream and downstream of a target gene region using two different sgRNAs, the region can be excised from the chromosome (excision KO). In this process, error-prone non-homologous end-joining (hereinafter may be referred to as “NHEJ”) is utilized. This method has also enabled excision of millions of base pairs, which was difficult using conventional gene targeting methods.

Further, by introducing an sgRNA, a Cas9 protein and a donor DNA into a fertilized egg at the same time, a cleavage site can be repaired by homology-directed repair (hereinafter may be referred to as “HDR”) using the donor DNA as a template, and a KI mouse can be obtained.

Previous studies have revealed that HDR activity is not observed in the G₁ phase, increases sharply in the S phase, and decreases in the G₂/M phase. It has also been found that NHEJ activity is observed throughout the cell cycle. Therefore, studies have been conducted to control the presence of Cas9 proteins in a cell cycle-specific manner to suppress the introduction of unintended insertion or deletion mutations (Indel) by NHEJ.

For example, it is known that the human Geminin protein is degraded during the G₁ phase. It is also known that the human Cdt1 protein is degraded during the S/G₂ phase. In addition. Non-Patent Document 1 describes the production of a fusion protein in which a part of the human Geminin protein is linked to Cas9 (hereinafter may be referred to as “SpCas9-hGem”) and a fusion protein in which a part of the human Cdt1 protein is linked to Cas9 (hereinafter may be referred to as “SpCas9-hCdt1”).

Non-Patent Document 1 also describes that SpCas9-hGem was degraded during the G₁ phase while SpCas9-hCdt1 was degraded during the S/G₂ phase (Non-Patent Document 1, FIG. 1C, etc.).

Non-Patent Document 1 further describes that the KI efficiency at the DNMT3B locus was 17.1% by the regular SpCas9, 16.5% by SpCas9-hGem, and 9.9% by SpCas9-hCdt1 (Non-Patent Document 1, FIG. 2C, etc.).

### CITATION LIST

### Patent Literature

Non-Patent Document 1: Howden S. E., et al., A Cas9 Variant for Efficient Generation of Indel-Free Knockin or Gene-Corrected Human Pluripotent Stem Cells, Stem Cell Reports, Vol. 7, 508-517, 2016.

### SUMMARY OF INVENTION

### Technical Problem

The production of KO mice and KI mice by genome editing of fertilized eggs is used in many research facilities because the cost and time are significantly reduced as compared with the method using ES cells. However, the production efficiency of excision KO mice and of HDR-dependent KI mice is not high. Therefore, an object of the present invention is to provide a modified Cas9 that improves KO efficiency and KI efficiency.

### Solution to Problem

The present invention includes the following aspects.

[1] A fusion protein including a Cas9 protein and a modifying peptide that modifies the Cas9 protein, in which the modifying peptide includes: a peptide composed of the amino acid sequence described in SEQ ID NO: 29; or a peptide composed of an amino acid sequence in which one or several amino acids are deleted, substituted, or added to the amino acid sequence described in SEQ ID NO: 29 and having activity to localize the Cas9 protein in the nucleus by forming a fusion protein with the Cas9 protein.

[2] The fusion protein according to [1], in which the modifying peptide includes: a peptide composed of the amino acid sequence described in SEQ ID NO: 1; or a peptide composed of an amino acid sequence in which one or several amino acids are deleted, substituted, or added to the amino acid sequence described in SEQ ID NO: 1 and having activity to localize the Cas9 protein in the nucleus by forming a fusion protein with the Cas9 protein.

[3] The fusion protein according to [1] or [2], in which the modifying peptide further includes: a peptide composed of the amino acid sequence described in SEQ ID NO: 2; or a peptide composed of an amino acid sequence in which one or several amino acids are deleted, substituted, or added to the amino acid sequence described in SEQ ID NO: 2 and having activity to degrade the Cas9 protein in the G₁ phase by forming a fusion protein with the Cas9 protein.

[4] The fusion protein according to any one of [1] to [3], in which the modifying peptide is provided at the N-terminal, at the C-terminal, or at a site that is neither the N-terminal nor the C-terminal of the Cas9 protein.

[5] A nucleic acid encoding the fusion protein according to any one of [1] to [4].

[6] A method for producing a cell in which the genomic DNA are edited, the method including contacting the fusion protein according to any one of [1] to [4] with the genomic DNA in a cell.

[7] The method according to [6], wherein the cell is a fertilized egg.

[8] A method of producing an animal in which the genomic DNA are edited, the method including contacting the fusion protein according to any one of [1] to [4] with the genomic DNA in a fertilized egg to obtain a fertilized egg in which the genomic DNA are edited and growing the fertilized egg in which the genomic DNA are edited into an individual to obtain the animal in which the genomic DNA are edited.

### Advantageous Effects of Invention

According to an embodiment of the present invention, a modified Cas9 that improves KO efficiency and KI efficiency can be provided.

### BRIEF DESCRIPTION OF DRAWINGS

FIG. 1A is a side-by-side view of the 1st to 110th amino acid sequence of the human Geminin protein and the 1st to 107th amino acid sequence of the mouse Geminin protein. FIG. 1B is a side-by-side view of the 30th to 120th amino acid sequence of the human Cdt1 protein and the 29th to 132nd amino acid sequence of the mouse Cdt1 protein. In FIG. 1C, the top row is a schematic diagram illustrating the structure of a pX330 vector, the middle row is a schematic diagram illustrating the structure of a pX330-mG vector, and the bottom row is a schematic diagram illustrating the structure of a pX330-mC vector.

FIGS. 2A to 2D are fluorescence micrographs presenting the results of immunostaining in Experiment Example 2.

FIG. 3A is a schematic diagram illustrating a method for excising the Tyr gene in Experiment Example 3. FIG. 3B and FIG. 3C are photographs presenting typical mice obtained in Experiment Example 3.

FIG. 4 is a schematic diagram illustrating a method for excising the Dmd gene in Experiment Example 4.

FIG. 5A is a schematic diagram illustrating the structure of a wild-type ROSA26 locus.

FIG. 5B is a schematic diagram illustrating the structure of a pRosa-CAG-fEGFP-Cables1 donor DNA plasmid used in Experiment Example 5. FIG. 5C is a schematic diagram illustrating the structure of a ROSA26 locus when a target knock-in occurs in Experiment Example 5.

FIG. 6A is a schematic diagram illustrating the structure of a wild-type Prdm14 locus.

FIG. 6B is a schematic diagram illustrating the structure of a pflox-Prdm14 donor DNA plasmid used in Experiment Example 6. FIG. 6C is a schematic diagram illustrating the structure of a Prdm14 locus when a target knock-in occurs in Experiment Example 6.

FIGS. 7A to 7C are fluorescence micrographs presenting the results of immunostaining in Experiment Example 7.

FIG. 8A is a schematic diagram illustrating the structure of a pX330-mC vector. FIG. 8B is a schematic diagram illustrating the structure of a pX330-pFmC vector. FIG. 8C is a schematic diagram illustrating the structure of a pX330-pCmC vector. FIG. 8D is a schematic diagram illustrating the structure of a pX330-pMmC vector. FIG. 8E is a schematic diagram illustrating the structure of a pX330-pNmC vector. FIG. 8F is a schematic diagram illustrating the structure of a pX330-pNNmC vector. FIG. 8G is a schematic diagram illustrating the structure of a pX330-pNCmC vector.

FIG. 9 shows fluorescence micrographs presenting the results of immunostaining in Experiment Example 9.

FIG. 10A is a photograph presenting the results of detecting each one of the Cas9 fusion proteins using an anti-FLAG antibody in Experiment Example 10. FIG. 10B is a photograph presenting the results of detecting each one of the Cas9 fusion proteins using an anti-Cas9 antibody in Experiment Example 10. FIG. 10C is a photograph presenting the results of detecting the nuclear protein PARP1 using an anti-PARP1 antibody in Experiment Example 10. FIG. 10D is a photograph presenting the results of detecting the cytoplasmic protein GAPDH using an anti-GAPDH antibody in Experiment Example 10.

FIG. 11 is a diagram summarizing the structure and nuclear localization of each one of the Cas9 fusion proteins based on the results of Experiment Examples 9 and 10.

FIG. 12 shows fluorescence micrographs presenting the results of immunostaining in Experiment Example 11.

### DESCRIPTION OF EMBODIMENTS

### Fusion Protein

In one embodiment, the present invention provides a fusion protein including a Cas9 protein and a modifying peptide that modifies the Cas9 protein, in which the modifying peptide includes: a peptide composed of the amino acid sequence described in SEQ ID NO: 1; or a peptide composed of an amino acid sequence in which one or several amino acids are deleted, substituted, or added to the amino acid sequence described in SEQ ID NO: 1 and having activity to localize the Cas9 protein in the nucleus by forming a fusion protein with the Cas9 protein.

Examples of the Cas9 protein include those derived from Streptococcus pyogenes, Staphylococcus aureus, Streptococcus thermophilus, and Geobacillus stearothermophilus. Of these. Cas9 derived from Streptococcus pyogenes (SpCas9) can be suitably used.

Examples of the modifying peptide that modifies the Cas9 protein include a peptide composed of the amino acid sequence described in SEQ ID NO: 1. A peptide composed of the amino acid sequence described in SEQ ID NO: 1 is a peptide composed of the 29th to 132nd amino acid sequence of the mouse Cdt1 protein.

Cdt1 is one of the licensing regulators ensuring that chromosomal replication is performed exactly once per cell cycle. Expression of Cdt1 is known to be higher in the G₁ phase and lower in the S phase due to ubiquitin-dependent degradation.

As described in Non-Patent Document 1, it is known that the abundance of fusion protein in which a part of the human Cdt1 protein is fused to Cas9 (SpCas9-Cdt1) shows cell cycle dependence and SpCas9-Cdt1 is degraded during the S/G₂ phase.

However, contrary to expectations, as described below in the Examples, a fusion protein according to an embodiment of the present invention in which a part of the mouse Cdt1 protein is fused to Cas9 (hereinafter may be referred to as “Cas9-mC”) did not show cell cycle dependence and was localized in the nucleus in all phases of the cell cycle. Localization to the nucleus was confirmed not only in mouse cells but also in human cells.

In addition, as described below in the Examples, it was revealed that genome editing using Cas9-mC enables highly efficient production of a large-scale genome-deficient mouse having from several tens of kilobase pairs to several megabase pairs of genomic deficiency and of knock-in mice and flox mice which are difficult to produce.

The modifying peptide is not limited to a peptide composed of the amino acid sequence described in SEQ ID NO: 1, and may have a mutation as long as the modifying peptide has the activity to localize the Cas9 protein in the nucleus by forming a fusion protein with the Cas9 protein.

Specifically, the peptide may be composed of an amino acid sequence in which one or several amino acids are deleted, substituted, or added to the amino acid sequence described in SEQ ID NO: 1. Here, “one or several” may be, for example, from 1 to 20, from 1 to 15, from 1 to 10, from 1 to 5, or from 1 to 3.

As described below in the Examples, the present inventors found that, as a modifying peptide that modifies the Cas9 protein, a peptide composed of the amino acid sequence described in SEQ ID NO: 29 also maintains the activity to localize the Cas9 protein in the nucleus. A peptide composed of the amino acid sequence described in SEQ ID NO: 29 is a peptide composed of the 29th to 80th amino acid sequence of the mouse Cdt1 protein.

A peptide composed of the amino acid sequence described in SEQ ID NO: 29 is shorter than a peptide composed of the amino acid sequence described in SEQ ID NO: 1. Therefore, the molecular weight of the fusion protein can be reduced. The size of the expression vector for the fusion protein can also be reduced. Thus, an easier-to-use fusion protein or nucleic acid encoding the fusion protein can be provided.

The modifying peptide is not limited to a peptide composed of the amino acid sequence described in SEQ ID NO: 29, and may have a mutation as long as the modifying peptide has the activity to localize the Cas9 protein in the nucleus by forming a fusion protein with the Cas9 protein.

Specifically, the peptide may be composed of an amino acid sequence in which one or several amino acids are deleted, substituted, or added to the amino acid sequence described in SEQ ID NO: 29. Here, “one or several” may be, for example, from 1 to 20, from 1 to 15, from 1 to 10, from 1 to 5, or from 1 to 3.

Hereinafter, a peptide composed of the amino acid sequence described in SEQ ID NO: 1, a peptide composed of an amino acid sequence in which one or several amino acids are deleted, substituted, or added to the amino acid sequence described in SEQ ID NO: 1, a peptide composed of the amino acid sequence described in SEQ ID NO: 29, or a peptide composed of an amino acid sequence in which one or several amino acids are deleted, substituted, or added to the amino acid sequence described in SEQ ID NO: 29 may be referred to as a “mouse Cdt1-derived peptide”.

In the fusion protein according to an embodiment of the present invention, the modifying peptide may further include a peptide composed of the amino acid sequence described in SEQ ID NO: 2. The amino acid sequence described in SEQ ID NO: 2 is a peptide composed of the 1st to 107th amino acid sequence of the mouse Geminin protein.

Geminin is a licensing inhibitor that inhibits the binding of a licensing factor to the replication initiation site of a genome whose replication has started once in the S phase. Expression of Geminin is known to be high in the S/G₂/M phases but decreases once the G₁ phase starts due to ubiquitin-dependent degradation.

As described below in the Examples, a fusion protein in which a part of the mouse Geminin protein is fused to Cas9 (hereinafter may be referred to as “Cas9-mG”) show a cell cycle dependence, is present in the cytoplasm from the S phase to the early G₂ phase, and is degraded from the late G₁ phase to the early S phase.

Thus, when the modifying peptide further includes a peptide composed of the amino acid sequence described in SEQ ID NO: 2, the modifying peptide can be degraded in the G₁ phase due to ubiquitin-dependent degradation. As a result, NHEJ activity in the G₁ phase can be suppressed, and the introduction of an unintended Indel mutation during a knock-in using genome editing can be suppressed.

The peptide further included by the modifying peptide is not limited to a peptide composed of the amino acid sequence described in SEQ ID NO: 2, and may have a mutation as long as the peptide has the activity to degrade the Cas9 protein in the G₁ phase by forming a fusion protein with the Cas9 protein.

Specifically, the peptide may be composed of an amino acid sequence in which one or several amino acids are deleted, substituted, or added to the amino acid sequence described in SEQ ID NO: 2. Here, “one or several” may be, for example, from 1 to 20, from 1 to 15, from 1 to 10, from 1 to 5, or from 1 to 3.

Hereinafter, a peptide composed of the amino acid sequence described in SEQ ID NO: 2, or a peptide composed of an amino acid sequence in which one or several amino acids are deleted, substituted, or added in the amino acid sequence described in SEQ ID NO: 2 may be referred to as a “Geminin-derived peptide”. The Geminin-derived peptide may have a part of the amino acid sequence of the human Geminin protein.

In the fusion protein of an embodiment according to the present invention, the position of the modifying peptide is not limited. For example, the position of the modifying peptide may be at the N-terminal or at the C-terminal, or at a site that is neither at the N-terminal nor the C-terminal of the Cas9 protein.

Further, in the fusion protein according to an embodiment of the present invention, the mouse Cdt1-derived peptide and the Geminin-derived peptide may be adjacent to each other or separated from each other. In addition, either the mouse Cdt1-derived peptide or the Geminin-derived peptide may be provided on the N-terminal side. That is, the Geminin-derived peptide may be provided on the C-terminal side of the mouse Cdt1-derived peptide, or the mouse Cdt1-derived peptide may be provided on the C-terminal side of the Geminin-derived peptide.

Also, when the positions of the mouse Cdt1-derived peptide and the Geminin-derived peptide are separated from each other, the mouse Cdt1-derived peptide and the Geminin-derived peptide may be provided at the N-terminal or at the C-terminal, or at a site that is neither at the N-terminal nor the C-terminal of the Cas9 protein, in a manner that is independent from each other.

For example, the Geminin-derived peptide may be provided at the N-terminal of the Cas9 protein while the mouse Cdt1-derived peptide may be provided at the C-terminal of the Cas9 protein. Alternatively, the mouse Cdt1-derived peptide may be provided at the N-terminal of the Cas9 protein while the Geminin-derived peptide may be provided at the C-terminal of the Cas9 protein.

Alternatively, either the mouse Cdt1-derived peptide or the Geminin-derived peptide may be provided at the N-terminal or the C-terminal of the Cas9 protein while the other is provided at the central portion of the Cas9 protein.

In the present specification, the N-terminal may include the vicinity of the N-terminal. Also, the C-terminal may include the vicinity of the C-terminal. That is, in some cases, even if the modifying peptide is not adjacent to the first amino acid from the N-terminal side of the Cas9 protein, when the modifying peptide is in the vicinity of the N-terminal of the Cas9 protein, the position of the modifying peptide is at the N-terminal of the Cas9 protein.

Similarly, in some cases, even if the modifying peptide is not adjacent to the first amino acid from the C-terminal side of the Cas9 protein, when the modifying peptide is in the vicinity of the C-terminal of the Cas9 protein, the position of the modifying peptide is at the C-terminal of the Cas9 protein.

Here, the vicinity may be, for example, a distance from 1 to 100 amino acids, a distance from 1 to 50 amino acids, a distance from 1 to 30 amino acids, or a distance from 1 to 20 amino acids.

The position of the modifying peptide may be a site that is neither at the N-terminal nor the C-terminal of the Cas9 protein. For example, the modifying peptide may be at the central portion of the Cas9 protein.

The fusion protein according to an embodiment of the present invention may include an additional peptide other than the Cas9 protein and the modifying peptide as long as the effect of the invention can be achieved. Examples of the additional peptide include: a tag peptide such as the FLAG tag, the His×6 tag, the MYC tag, the HA tag, and the V5 tag; a fluorescent protein such as the green fluorescent protein (GFP) and GFP derivatives; a peptide introduced in the process of genetic recombination, such as a peptide derived from the multi-cloning site of a vector and a peptide derived from part of a primer; and a nuclear localization signal (NIS) originally incorporated in a vector.

In the present specification, the terms “protein” and “peptide” are used without strict distinction and may be referring to a peptide or a protein depending on the number of amino acids.

### Nucleic Acid

One embodiment according to the present invention provides a nucleic acid encoding the fusion proteins described above. Introduction of the nucleic acid according to the embodiment into a cell can express the fusion protein described above. In addition, an mRNA encoding the fusion protein described above can be obtained by transcribing the nucleic acid according to the embodiment in an in vitro transcription system.

The nucleic acid according to the embodiment may be an expression vector. The expression vector is not limited, and examples include a transposon vector, a viral vector, an episomal vector, and a plasmid vector.

Method for Producing Cell in which Genomic DNA are Edited One embodiment according to the present invention provides a method for producing a cell in which the genomic DNA are edited, the method including contacting the fusion protein described above with genomic DNA in a cell.

The fusion protein described above is contacted with the genomic DNA along with the gRNA corresponding to a target sequence. More specifically, the fusion proteins described above can be contacted with the genomic DNA in a cell by being introduced into the cell along with the gRNA. This can result in the formation of a DNA double-strand break (DSB) in the target sequence portion.

Examples of the cell include an ES cell, an iPS cell, a fertilized egg, and a cultured cell. Examples of the cultured cell include an animal cell, an insect cell, a plant cell, and a yeast cell.

When the cell is a fertilized egg, a fertilized egg in which the genomic DNA are edited can be obtained. By growing the fertilized egg into an individual, an animal in which the genomic DNA are edited can be obtained. The animal is not limited, and examples include: a mammal such as a mouse, a rat, a sheep, a pig, a monkey, and a human; a bird such as a chicken; an amphibian such as a Xenopus laevis, a reptile such as a gecko, a fish such as a zebrafish and an Oryzias latipes, and an insect such as a Bombyx mori.

The introduction of the fusion protein and the gRNA into a cell can be accomplished by lipofection, microinjection, electroporation, and the like.

The gRNA may be a complex of a CRISPR RNA (crRNA) and a trans-activating CRISPR RNA (tracrRNA), or may be a single gRNA (sgRNA) that is a combination of a tracrRNA and a crRNA.

The gRNA can be introduced into the cell in the form of an RNA or in the form of an expression vector. Examples of a method for preparing the gRNA in the form of an RNA include: a method for synthesizing a gRNA by an in vitro transcription reaction using a construct in which a promoter such as T7 is added upstream of a nucleic acid fragment encoding the gRNA, and a method for chemically synthesizing a gRNA. When chemically synthesizing the gRNA, a chemically modified RNA may be used.

When preparing the gRNA in the form of an expression vector, the expression vector may be a plasmid vector or a viral vector that transcribes the gRNA from a Pol III promoter such as the Ha promoter or the U6 promoter. When the gRNA is expressed using an expression vector, the gRNA may be expressed constitutively or under the control of an expression-inducing promoter.

The fusion protein may be introduced into a donor cell in the form of an expression vector expressed from a Pol II promoter or in the form of a purified protein. Examples of the expression vector as the fusion protein include a transposon vector, a viral vector, an episomal vector, and a plasmid vector.

The Indel mutation may be introduced in the process in which a formed DSB is repaired by NHEJ. Alternatively, by simultaneously cleaving upstream and downstream of a target gene region using two different sgRNAs, the region can be excised from the chromosome.

A donor DNA may also be introduced into the cell along with the fusion protein and the gRNA. The donor DNA preferably includes a 5′ homologous arm region, a target nucleic acid sequence region, and a 3′ homologous arm region. In this case, the DSB can be repaired by homology-directed repair (HDR) using the donor DNA as a template to knock in the target nucleic acid sequence region in the donor DNA. Alternatively, the donor DNA can be knocked in by an HDR-independent method such as the PITCh method or the HITI method.

Method for Producing Animal in which Genomic DNA are Edited

One embodiment according to the present invention provides a method of producing an animal in which the genomic DNA are edited, the method including contacting the fusion protein described above with the genomic DNA in a fertilized egg to obtain a fertilized egg in which the genomic DNA are edited and growing the fertilized egg in which the genomic DNA are edited into an individual to obtain the animal in which the genomic DNA are edited.

The steps of obtaining a fertilized egg in which the genomic DNA are edited are the same as those in the above-mentioned method for producing a cell in which the genomic DNA are edited when the cell is a fertilized egg. By growing the fertilized egg into an individual, an animal in which the genomic DNA are edited can be obtained. Examples of the animal include those described above.

The method for growing the fertilized egg in which the genomic DNA are edited into an individual can be selected depending on the species of the animal. For example, in the case of a mammal, the fertilized egg in which the genomic DNA are edited can be grown into an individual by being transplanted into the oviduct or uterus of a surrogate mother. In the case of a bird, an amphibian, a reptile, a fish, an insect, and the like, the fertilized egg in which the genomic DNA are edited can be grown into an individual by being cultured and developed in an appropriate environment.

### EXAMPLES

Hereinafter, the present invention will be described in further detail with reference to examples, but the present invention is not limited to the following examples.

### Materials and Methods

### Mouse

C57BL/J mice were purchased from Charles River Laboratories Japan, Inc. ICR mice (available from Charles River Laboratories Japan, Inc.) were used as pseudopregnant mice for producing mice. Mice were housed at a room temperature of 23.5° C.±2.5° C. and a humidity of 52.5°±12.5% under a light-dark cycle of 14:10 hours.

### Production of Mice

Female C57BL/6J mice at from 12 to 20 weeks of age were subcutaneously administered with 5 units of pregnant mare serum gonadotropin, and after 48 hours, intraperitoneally administered with 5 units of human chorionic gonadotropin and mated with male C57BL/6J mice. Mating was confirmed by the presence of vaginal plugs, and fertilized eggs at the pronuclear stage were collected from the oviducts. No frozen fertilized eggs or in vitro fertilized eggs were used. pX330 vector, pX330-mG vector, and pX330-mC vector were diluted to 5 ng/μl with sterilized distilled water and filtered through a PVDF membrane having a pore size of 0.22 μm before use. Various donor vectors were diluted to 10 ng/μl with sterilized distilled water and filtered through a PVDF membrane having a pore size of 0.22 μm before use. Each one of the vectors was microinjected into the male pronuclei of the pronuclear embryos. Fertilized eggs that survived from 15 minutes to 2 hours after the microinjection were transplanted into the oviducts of the pseudopregnant mice and developed into individuals.

### Analysis of Genotype

Genomic DNA was extracted from the tip of the tail of a mouse having a size of 0.5 mm or less and purified using PI-200, a genomic extraction device, and used for genotype analysis. Confirmation of the KI allele and the flox allele was performed by PCR using a PrimeSTAR GXL DNA Polymerase (available from Takara Bio Inc.). Confirmation of the KO allele was performed by PCR using an AmpliTaq Gold 360 Master Mix (available from Thermo Fisher Scientific Inc.).

### Experiment Example 1

### Production 1 of Cas9 Fusion Protein Expression Vector

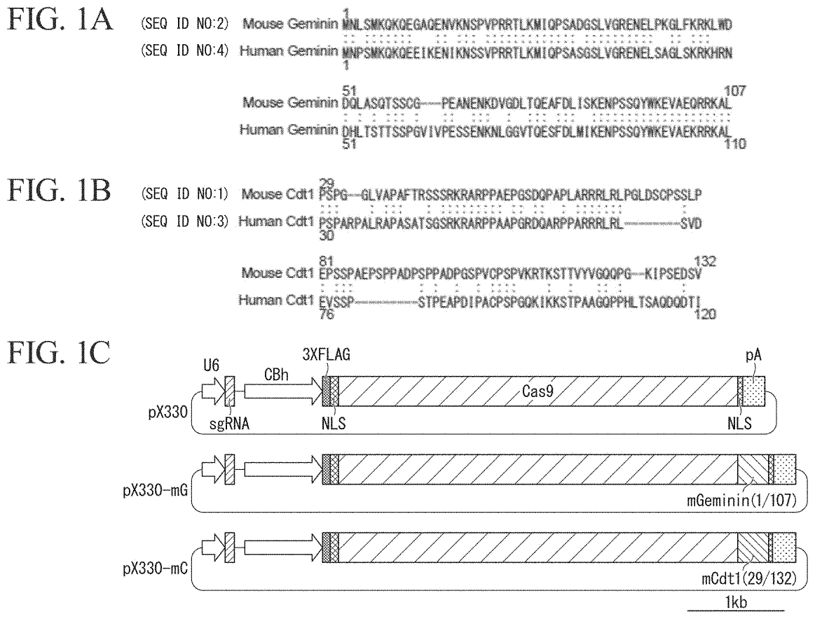

pX330 (Plasmid #42230, available from Addgene) is a vector capable of expressing both sgRNA and Cas9. The top row of FIG. 1C illustrates the structure of the pX330 vector. In the pX330 vector, sgRNA is expressed by the U6 promoter. Furthermore, the SpCas9 protein is expressed by the CBh promoter. The N-terminal and the C-terminal of the SpCas9 protein are each linked to a nuclear localization signal (NLS) derived from the SV40 virus. In addition, the N-terminal side of the NLS at the N-terminal is linked to a 3×FLAG tag peptide.

A Cas9 fusion protein expression vector was prepared based on the pX330 vector. More specifically, a vector (SEQ ID NO: 5, hereinafter may be referred to as “pX330-mG”) expressing a fusion protein (hereinafter may be referred to as “Cas9-mG”) in which the C-terminal of the SpCas9 protein was linked to a peptide composed of the 1st to 107th of a Geminin protein derived from a mouse rather than a human was prepared.

FIG. 1A is a side-by-side view of the 1st to 110th amino acid sequence of the human Geminin protein (SEQ ID NO: 4) and the 1st to 107th amino acid sequence of the mouse Geminin protein (SEQ ID NO: 2). Also, the structure of the pX330-mG vector is illustrated in the middle row of FIG. 1C.

In addition, a vector (SEQ ID NO: 6, hereinafter may be referred to as “pX330-mC”) expressing a fusion protein (hereinafter may be referred to as “Cas9-mC”) in which the C-terminal of the SpCas9 protein was linked to a peptide composed of the 29th to 132nd of a Cdt1 protein derived from a mouse rather than a human was prepared.

FIG. 1B is a side-by-side view of the 30th to 120th amino acid sequence of the human Cdt1 protein (SEQ ID NO: 3) and the 29th to 132nd amino acid sequence of the mouse Cdt1 protein (SEQ ID NO: 1). Also, the structure of the pX330-mC vector is illustrated in the bottom row of FIG. 1C.

### Experiment Example 2

### Study of Cell Cycle Dependence and Subcellular Localization of Cas9, Cas9-mG, and Cas9-mC

Fertilized mouse eggs were used to study cell cycle dependence and subcellular localization of Cas9-mG protein and Cas9-mC protein expressed by the introduction of the expression vectors prepared in Experiment Example 1.

First, using an in vitro transcription system. Cas9-mG mRNA, Cas9-mC mRNA, and Cas9 mRNA were prepared separately by transcribing each one of the expression vectors prepared in Experiment Example 1.

Then, the Cas9-mG mRNA and the Cas9-mC mRNA were each injected into the cytoplasm of fertilized mouse eggs obtained via intracytoplasmic sperm injection. For comparison, the same study was conducted on both fertilized mouse eggs injected with regular Cas9 mRNA and fertilized mouse eggs injected with nothing.

As described above, the Cas9-mG protein, the Cas9-mC protein and the regular Cas9 protein encoded by each one of the vectors prepared in Experiment Example 1 are each linked to a 3×FLAG tag peptide. Therefore, the Cas9-mG protein, the Cas9-mC protein and the regular Cas9 protein translated from the Cas9-mG mRNA, the Cas9-mC mRNA and the Cas9 mRNA injected into the fertilized mouse eggs in this Experiment Example are also each linked to a 3×FLAG tag peptide. Thus, each one of the Cas9 proteins can be detected by immunostaining using an anti-FLAG antibody.

The fertilized eggs were then cultured in vitro, and the 2-cell stage embryos from the late G₁ phase to the early S phase and the 2-cell stage embryos from the late S phase to the early G₂ phase were immunostained with the anti-FLAG antibody and observed by fluorescence microscopy.

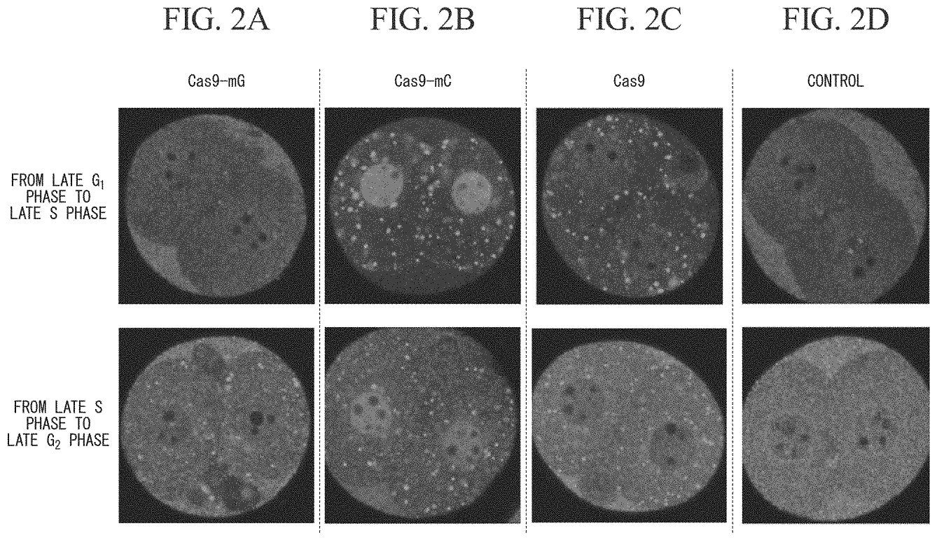

FIGS. 2A to 2D are fluorescence micrographs presenting the results of immunostaining. FIG. 2A is the result of the Cas9-mG protein, FIG. 2B is the result of the Cas9-mC protein, FIG. 2C is the result of the regular Cas9 protein, and FIG. 2D is a control in which nothing is introduced.

As a result, the regular Cas9 protein was detected as a dot-like signal in the cytoplasm at any phase of the cell cycle. As described above, the N-terminal and the C-terminal of the Cas9 protein used in this Experimental Example are each linked to a nuclear localization signal derived from the SV40 virus; however, no nuclear localization of the Cas9 protein was observed.

Meanwhile, it was confirmed that the Cas9-mG protein was not detected in the late G₁ phase to the early S phase and was degraded. The Cas9-mG protein was also detected as a dot-like signal in the cytoplasm from the S phase to the early G₂ phase. As described above, the N-terminal and the C-terminal of the Cas9-mG protein used in this Experimental Example are each linked to a nuclear localization signal derived from the SV40 virus; however, no nuclear localization of the Cas9 protein was observed.

In addition, contrary to expectations, cell cycle dependence was not confirmed for the Cas9-mC protein, and signals were detected in all phases of the cell cycle. Furthermore, the nuclei were stained strongly in all phases of the cell cycle. This result revealed that the Cas9-mC protein showed high nuclear localization.

### Experiment Example 3

### Production 1 of Large-Scale Genome-Deficient Mouse Using Cas9-mC

Investigation regarding whether Cas9-mC would increase the efficiency of excision of large-scale genomic regions was conducted.

Specifically, Cas9-mC was used to produce mice in which the Tyr gene was fully excised (excision KO mice). Also, for comparison, mice in which the Tyr gene was fully excised were prepared by the same method except that the regular Cas9 was used. The Tyr gene is a gene that dominates the fur color of a mouse, and a mouse lacking both alleles of the Tyr gene exhibits the albino trait. Note that a mouse lacking one allele of the Tyr gene does not exhibit the albino trait.

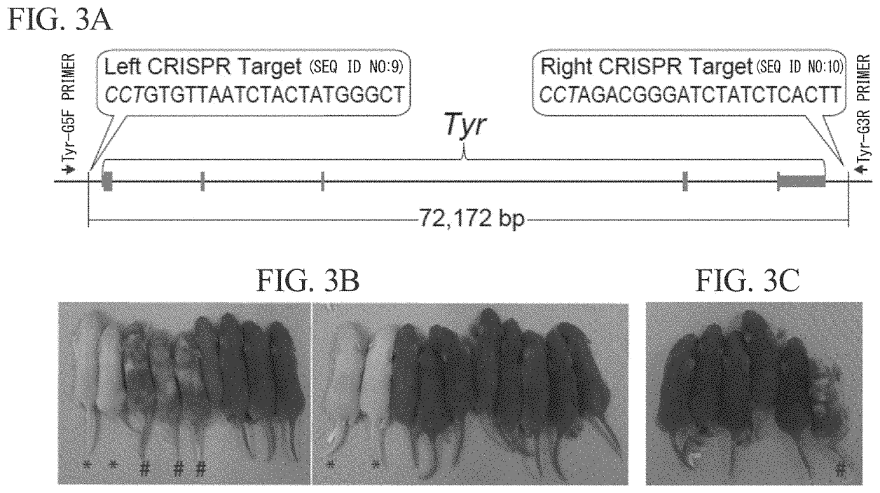

FIG. 3A is a schematic diagram illustrating a method for excising the Tyr gene. In FIG. 3A. “Tyr-G5F primer” (SEQ ID NO: 7) and “Tyr-G3R primer” (SEQ ID NO: 8) are the primers used to confirm the genotype of a mouse.

Specifically, a pX330-mC-Tyr-L vector that expresses an sgRNA targeting the genomic sequence upstream of the Tyr gene (Left CRISPR Target, SEQ ID NO: 9) and the Cas9-mC protein, as well as a pX330-mC-Tyr-R vector that expresses an sgRNA targeting the genomic sequence downstream of the Tyr gene (Right CRISPR Target, SEQ ID NO: 10) and the Cas9-mC protein were prepared.

Then, a pX330-Tyr-L vector that expresses an sgRNA targeting the genomic sequence upstream of the Tyr gene (Left CRISPR Target, SEQ ID NO: 9) and the regular Cas9 protein, as well as a pX330-Tyr-R vector that expresses an sgRNA targeting the genomic sequence downstream of the Tyr gene (Right CRISPR Target, SEQ ID NO: 10) and the regular Cas9 protein were prepared. The distance between the Left CRISPR Target (SEQ ID NO: 9) and the Right CRISPR Target (SEQ ID NO: 10) was 72,172 base pairs.

Then, the male pronuclei of fertilized eggs (at the pronuclear stage) of C57BL6/J mice with black fur obtained by natural mating were respectively microinjected with a mixture of the pX330-mC-Tyr-L vector (5 ng/μL) and the pX330-mC-Tyr-R vector (5 ng/μL) and a mixture of the pX330-Tyr-L vector (5 ng/μL) and the pX330-Tyr-R vector (5 ng/μL). Subsequently, the fertilized eggs with the vectors introduced were transplanted into the oviducts of pseudopregnant mice and developed into mice, and the fur color of the resulting mice was observed.

FIGS. 3B and 3C are photographs presenting typical mice obtained. FIG. 3B is a photograph of mice developed from fertilized eggs with Cas9-mC introduced, while FIG. 3C is a photograph of mice developed from fertilized eggs with regular Cas9 introduced. In FIGS. 3B and 3C, “*” indicates mice showing a complete albino trait, while “#” indicates mice showing a mosaic albino trait.

It is believed that a complete albino trait is the result of a full-length deletion of the Tyr gene in both alleles in perhaps the 1-cell stage of a fertilized egg, while a mosaic albino trait is the result of a full-length deletion of the Tyr gene in both alleles of either cell from the 2-cell stage onward of a fertilized egg.

As a result, among the 34 mice obtained from the fertilized eggs with Cas9-mC introduced, 5 showed the complete albino trait while 3 showed the mosaic albino trait. In addition, analysis of the genotype by PCR revealed that 17 (50.0%) of the 34 mice developed from the fertilized eggs with Cas9-mC introduced had alleles with the Tyr gene completely excised.

Further, among the 21 mice obtained from the fertilized eggs with the regular Cas9 introduced, 1 showed the mosaic albino trait while none showed the complete albino trait. In addition, analysis of the genotype by PCR revealed that 6 (28.6%) of the 21 mice developed from the fertilized eggs with the regular Cas9 introduced had alleles with the Tyr gene completely excised. Table 1 below shows the results of producing the large-scale Tyr-deficient mice.

The above results show that by using Cas9-mC, a large-scale genome-deficient mouse having several tens of kilobase pairs of genomic deficiency can be produced with high efficiency.

TABLE 1

| | Cas9-mC | Cas9 |
|---|---|---|
| Number of fertilized eggs with veetors introduced | 102 | 107 |
| Number of fertilized eggs transplanted into | 100 | 100 |
| pseudopregnant mice | | |
| Total number of newborns | 34 | 21 |
| Number of newborns with large-scale deficient | 17 | 6 |
| alleles | (50.0%)^{a} | (28.6%)^{a} |
| Number of newborns showing complete albino | 5 | 0 |
| trait | | |
| Number of newborns showing mosaic albino trait | 3 | 1 |

^{a}Number of newborns with large-scale deficient allele/Total number of newborns × 100

### Experiment Example 4

### Production 2 of Large-Scale Genome-Deficient Mouse Using Cas9-mC

Investigation regarding whether Cas9-mC would increase the efficiency of excision of large-scale genomic regions was conducted.

Specifically. Cas9-mC was used to produce mice in which the Dmd gene was fully excised (excision KO mice). The Dmd gene is known to be the longest gene encoding a protein. Also, for comparison, mice in which the Dmd gene was fully excised were prepared by the same method except that regular Cas9 was used. Because the Dmd gene is located on the X chromosome, the male has 1 copy and the female has 2 copies.

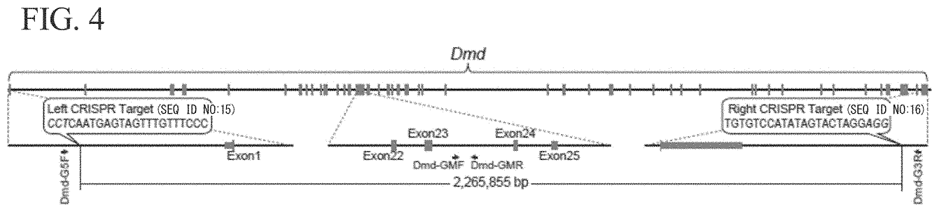

FIG. 4 is a schematic diagram illustrating a method for excising the Dmd gene. In FIG. 4, “Dmd-G5F” (SEQ ID NO: 11), “Dmd-GMF” (SEQ ID NO: 12), “Dmd-GMR” (SEQ ID NO: 13), and “Dmd-G3R” (SEQ ID NO: 14) are the primers used to confirm the genotype of a mouse.

Specifically, first, a pX330-mC-Dmd-L vector that expresses an sgRNA targeting the genomic sequence upstream of the Dmd gene (Left CRISPR Target, SEQ ID NO: 15) and the Cas9-mC protein, as well as a pX330-mC-Dmd-R vector that expresses an sgRNA targeting the genomic sequence downstream of the Dmd gene (Right CRISPR Target, SEQ ID NO: 16) and the Cas9-mC protein were prepared.

Then, a pX330-Dmd-L vector that expresses an sgRNA targeting the genomic sequence upstream of the Dmd gene (Left CRISPR Target. SEQ ID NO: 15) and the regular Cas9 protein, as well as a pX330-Dmd-R vector that expresses an sgRNA targeting the genomic sequence downstream of the Dmd gene (Right CRISPR Target, SEQ ID NO: 16) and the regular Cas9 protein were prepared. The distance between the Left CRISPR Target (SEQ ID NO: 15) and the Right CRISPR Target (SEQ ID NO: 16) was 2,265,855 base pairs.

Then, the male pronuclei of fertilized eggs (at the pronuclear stage) of C57BL6/J mice obtained by natural mating were respectively microinjected with a mixture of the pX330-mC-Dmd-L vector (5 ng/μL) and the pX330-mC-Dmd-R vector (5 ng/L) and a mixture of the pX330-Dmd-L vector (5 ng/μL) and the pX330-Dmd-R vector (5 ng/μL). Subsequently, the fertilized eggs with the vectors introduced were transplanted into the oviducts of pseudopregnant mice and developed into mice, of which the genotypes were confirmed by PCR.

As a result, a total of 68 mice were obtained from the fertilized eggs with Cas9-mC introduced. Furthermore, analysis of the genotype by PCR revealed that 11 (16.2%) of the 68 mice developed from the fertilized eggs with Cas9-mC introduced had alleles with the Dmd gene completely excised.

In addition, a total of 70 mice were obtained from the fertilized eggs with the regular Cas9 induced. Furthermore, analysis of the genotype by PCR revealed that 6 (8.6%) of the 70 mice developed from the fertilized eggs with the regular Cas9 introduced had alleles with the Dmd gene completely excised.

It is worth noting that the 6 mice with the full Dmd excision knock-out alleles obtained by the introduction of regular Cas9 were mosaic or heterozygous variants that also retained other Dmd alleles, while 5 male mice and 1 female mouse with the full Dmd excision knock-out alleles obtained by the introduction of Cas9-mC were individuals with full deletion of the Dmd gene having only the full Dmd knock-out allele. Table 2 below shows the results of producing the large-scale Dmd-deficient mice.

The above results show that by using Cas9-mC, a large-scale genome-deficient mouse having several megabase pairs of genomic deficiency can be produced with high efficiency.

TABLE 2

| | Cas9-mC | Cas9 |
|---|---|---|
| Number of fertilized eggs with vectors | 165 | 166 |
| introduced | | |
| Number of fertilized eggs transplanted into | 149 | 154 |
| pseudopregnant mice | | |
| Total number of newborns | 68 | 70 |
| Number of newborns with large-scale deficient | 11 | 6 |
| alleles | (16.2%)^{a} | (8.6%)^{a} |
| Number of newborns with only large-scale | 6 | 0 |
| deficient alleles | (♂: 5, ♀: 1) | |

^{a}Number of newborns with large-scale deficient alleles/Total number of newborns × 100

### Experiment Example 5

### Production 1 of Knock-in Mouse Using Cas9-mC

In genome editing of fertilized eggs, it is known that it is difficult to produce a mouse knocked in with a gene fragment including a base sequence having a high GC content and a flox mouse in which LoxP sequences must be introduced into two sites at the same time. Investigation regarding whether Cas9-mC is useful for producing such knock-in mice or flox mice which are difficult to produce was conducted.

First, an experiment was conducted in which an expression cassette CAG-flox EGFP-Cables1 including a Cables1 gene cDNA having a CAG promoter containing a base sequence with a high GC content and a base sequence with a high GC content were knocked in. The locus of knock-in was also a ROSA26 locus in which the 5′ homologous arm region includes a base sequence having a high GC content.

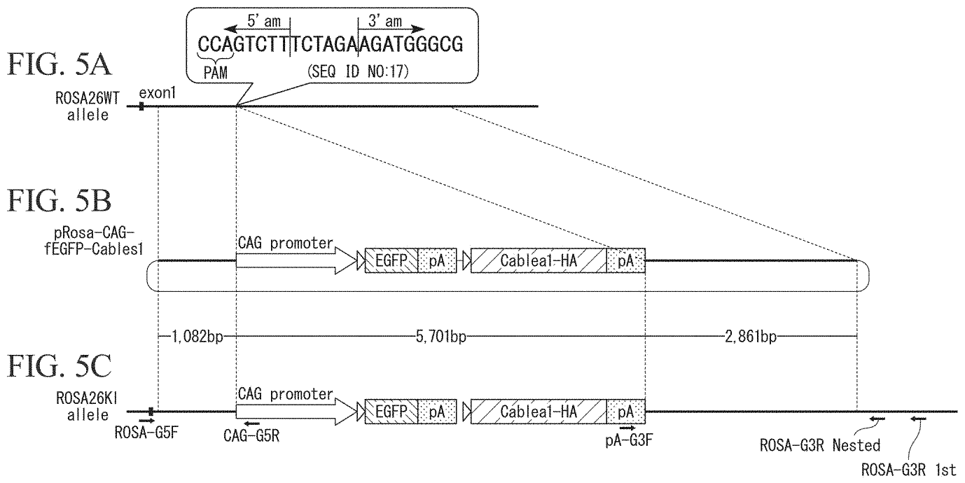

FIGS. 5A to 5C are schematic diagrams illustrating a method for knocking a CAG-flox EGFP-Cables1 gene fragment into the ROSA26 locus. FIG. 5A is a schematic diagram illustrating the structure of a wild-type ROSA26 locus. FIG. 5A also illustrates the positions of the target sequence of sgRNA (SEQ ID NO: 17), 5′ homologous arm region and 3′ homologous arm region.

FIG. 5B is a schematic diagram illustrating the structure of a pRosa-CAG-fEGFP-Cables1 donor DNA plasmid. FIG. 5C is a schematic diagram illustrating the structure of the ROSA26 locus when the target knock-in occurs. In FIG. 5C, “ROSA-G5F” (SEQ ID NO: 18) “CAG-G5R” (SEQ ID NO: 19), “pA-G3F” (SEQ ID NO: 20), “ROSA-G3R Nested” (SEQ ID NO: 21), and “ROSA-G3R 1st” (SEQ ID NO: 22) are the primers used to confirm the genotype of a mouse.

Specifically, first, a pX330-mC-ROSA vector that expresses an sgRNA targeting the first intron of the ROSA26 locus and the Cas9-mC protein, as well as a pX330-ROSA vector that expresses an sgRNA targeting the first intron of the ROSA26 locus and the regular Cas9 protein were prepared.

Then, the male pronuclei of fertilized eggs (at the pronuclear stage) of C57BL6/J mice obtained by natural mating were respectively microinjected with a mixture of the pX330-mC-ROSA vector (5 ng/μL) and the pRosa-CAG-fEGFP-Cables1 vector (10 ng/μL) and a mixture of the pX330-ROSA vector (5 ng/μL) and the pRosa-CAG-fEGFP-Cables1 vector (10 ng/μL). Subsequently, the fertilized eggs with the vectors introduced were transplanted into the oviducts of pseudopregnant mice and developed into mice, of which the genotypes were confirmed by PCR.

As a result, a total of 19 mice were obtained from the fertilized eggs with Cas9-mC introduced. Furthermore, analysis of the genotype by PCR revealed that 4 (21.1%) of the 19 mice developed from the fertilized eggs with Cas9-mC introduced had the target knock-in alleles.

In addition, a total of 44 mice were obtained from the fertilized eggs into which the regular Cas9 was introduced, but analysis of the genotype by PCR revealed that no mouse having the target knock-in alleles was obtained. Table 3 below shows the results of producing the knock-in mice.

The above results indicate that by using Cas9-mC, it is possible to produce KI mice and flox mice which are difficult to produce.

TABLE 3

| | Cas9-mC | Cas9 |
|---|---|---|
| Number of fertilized eggs with vectors introduced | 165 | 122 |
| Number of fertilized egs transplanted into | 154 | 106 |
| pseudopregnant mice | | |
| Total number of newborns or embryos^{b} of which | 19 | 44 |
| the genotype was analyzed | | |
| Number of newborns or embryos^{b} with the knock- | 4 | 0 |
| in alleles | (21.1%)^{a} | (0.0%)^{a} |

^{a}Number of newborns or embryos with the knock-in alleles/Total number of newborns or embryos of which the genotype was analyzed × 100
^{b}Embryo that is 18.5 days after fertilization

### Experiment Example 6

### Production 2 of Knock-in Mouse Using Cas9-mC

Then, LoxP sequences were introduced upstream and downstream of the sixth exon of the Prdm14 gene to produce a Prdm14 flox mouse.

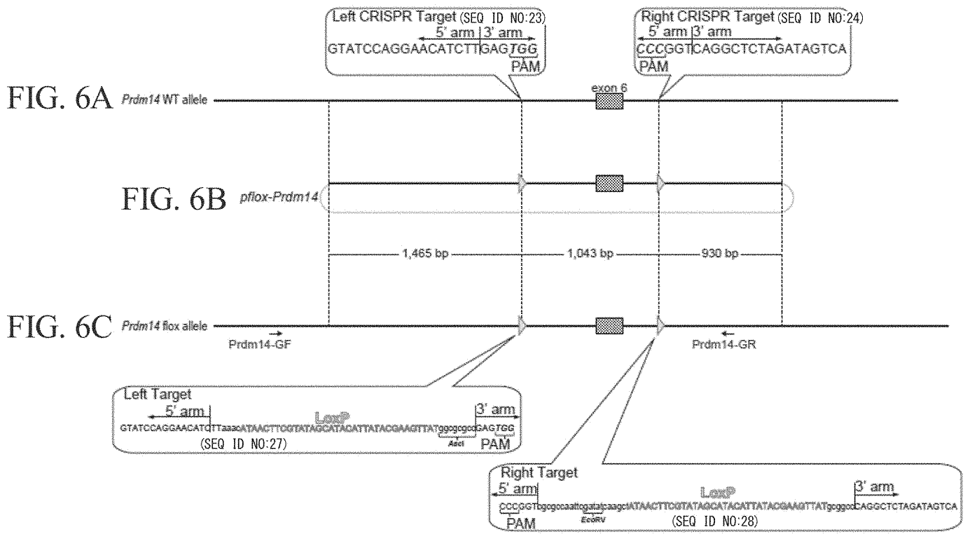

FIGS. 6A to 6C are schematic diagrams illustrating a method for knocking the LoxP sequences into the Prdm14 locus. FIG. 6A is a schematic diagram illustrating the structure of a wild-type Prdm14 locus.

FIG. 6A also illustrates the positions of the sgRNA targeting the genomic sequence upstream of the sixth exon of the Prdm14 gene (Left CRISPR Target. SEQ ID NO: 23), the sgRNA targeting the genomic sequence downstream of the sixth exon of the Prdm14 gene (Right CRISPR Target, SEQ ID NO: 24), the 5′ homologous arm region and the 3′ homologous arm region.

FIG. 6B is a schematic diagram illustrating the structure of a pflox-Prdm14 donor DNA plasmid. FIG. 6C is a schematic diagram illustrating the structure of the Prdm14 locus when the target knock-in occurs. In FIG. 6C, “Prdm14-GF” (SEQ ID NO: 25) and “Prdm14-GR” (SEQ ID NO: 26) are the primers used to confirm the genotype of a mouse, while “LoxP” represents the LoxP sequence knocked in.

Specifically, first, a pX330-mC-Prdm14-L vector that expresses an sgRNA targeting the genomic sequence upstream of the Prdm14 gene (Left CRISPR Target. SEQ ID NO: 23) and the Cas9-mC protein, as well as a pX330-mC-Prdm14-R vector that expresses an sgRNA targeting the genomic sequence downstream of the Prdm14 gene (Right CRISPR Target. SEQ ID NO: 24) and the Cas9-mC protein were prepared.

Then, a pX330-Prdm14-L vector that expresses an sgRNA targeting the genomic sequence upstream of the Prdm14 gene (Left CRISPR Target. SEQ ID NO: 23) and the regular Cas9 protein, as well as a pX330-Prdm14-R vector that expresses an sgRNA targeting the genomic sequence downstream of the Prdm14 gene (Right CRISPR Target. SEQ ID NO: 24) and the regular Cas9 protein were prepared.

Then, the male pronuclei of fertilized eggs (at the pronuclear stage) of C57BL6/J mice obtained by natural mating were respectively microinjected with a mixture of the pX330-mC-Prdm14-L vector (5 ng/μL), the pX330-mC-Prdm14-R vector (5 ng/μL), and the pflox-Prdm14 vector (10 ng/μL), as well as a mixture of the pX330-Prdm14-L vector (5 ng/μL), the pX330-Prdm14-R vector (5 ng/μL), and the pflox-Prdm14 vector (10 ng/μL). Subsequently, the fertilized eggs with the vectors introduced were transplanted into the oviducts of pseudopregnant mice and developed into mice, of which the genotypes were confirmed by PCR.

As a result, a total of 22 mice were obtained from the fertilized eggs with Cas9-mC introduced. Furthermore, analysis of the genotype by PCR revealed that 4 (18.2%) of the 22 mice developed from the fertilized eggs with Cas9-mC introduced had the target knock-in alleles.

In addition, a total of 25 mice were obtained from the fertilized eggs into which the regular Cas9 was introduced, but analysis of the genotype by PCR revealed that no mouse having the target knock-in alleles was obtained. Table 4 below shows the results of producing the knock-in mice.

TABLE 4

| | Cas9-mC | Cas9 |
|---|---|---|
| Number of fertilized eggs with vectors introduced | 104 | 104 |
| Number of fertilized eggs transplanted into | 87 | 85 |
| pseudopregnant mice | | |
| Number of newborns grown until weaning | 22 | 25 |
| Number of newborns with the knock-in alleles | 4 | 0 |
| | (18.2%)^{a} | (0.0%)^{a} |

^{a}Number of newborns with the knock-in alleles/Total number of newborns grown until weaning × 100

### Experiment Example 7

### Study of Subcellular Localization of Cas9, Cas9-mG, Cas9-mC

HEK293T cells, which are cells derived from human fetal kidney, were used to study subcellular localization of the Cas9-mG protein and the Cas9-mC protein expressed by the introduction of the expression vectors prepared in Experiment Example 1.

First, the pX330-mG vector and the pX330-mC vector were introduced into HEK293T cells, respectively. HEK293T cells with the pX330 vector introduced were also prepared for comparison.

As described above, the Cas9-mG protein, the Cas9-mC protein, and the regular Cas9 protein encoded by each one of the vectors are each linked to a 3×FLAG tag peptide. Thus, each one of the Cas9 proteins can be detected by immunostaining using an anti-FLAG antibody.

Each cell was then formaldehyde-fixed and immunostained with an anti-FLAG antibody. The nuclei were stained with 4′,6-diamidino-2-phenylindole (DAPI). Each stained cell was then observed using a fluorescence microscope.

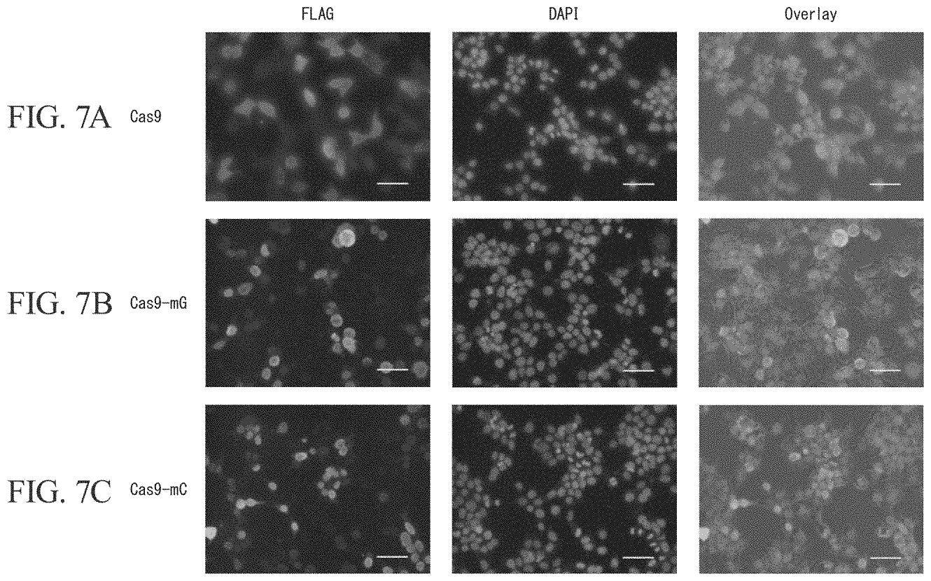

FIGS. 7A to 7C are fluorescence micrographs presenting the results of immunostaining. FIG. 7A is the result of the regular Cas9 protein, FIG. 7B is the result of the Cas9-mG protein, and FIG. 7C is the result of the Cas9-mC protein. In FIG. 7, “Overlay” is the result of synthesizing the staining result using the anti-FLAG antibody and the staining result using DAPI.

As a result, the regular Cas9 protein was detected in the cytoplasm. As described above, the N-terminal and the C-terminal of the Cas9 protein used in this Experimental Example are each linked to a nuclear localization signal derived from the SV40 virus; however, no nuclear localization of the Cas9 protein was observed.

Meanwhile, the Cas9-mG protein was found to be localized in the nucleus. The Cas9-mC protein was also found to be localized in the nucleus. These results revealed that the Cas9-mC protein showed high nuclear localization even in human cells.

### Experiment Example 8

### Production 2 of Cas9 Fusion Protein Expression Vector

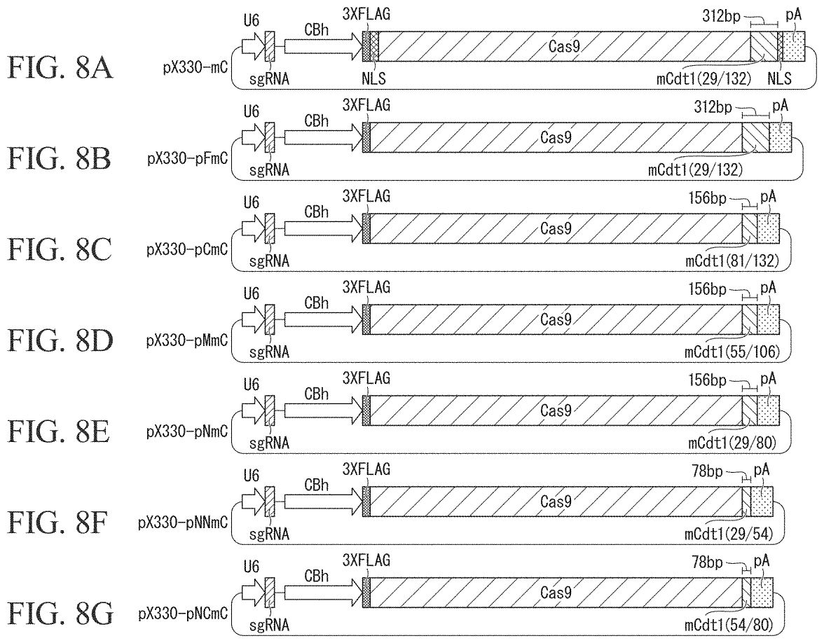

The N-terminal and the C-terminal of the SpCas9 protein expressed by the pX330-mC vector prepared in Experiment Example 1 each had a nuclear transition signal (NLS) derived from the SV40 virus. FIG. 8A illustrates the structure of the pX330-mC vector.

In this Experiment Example, a vector (SEQ ID NO: 30, hereinafter may be referred to as “pX330-pFmC”) that removes these NLS present at the N-terminal and the C-terminal of the SpCas9 protein and that expresses a fusion protein (hereinafter may be referred to as “Cas9-pFmC”) in which the C-terminal of the SpCas9 protein was linked to a peptide composed of the 29th to 132nd of the Cdt1 protein derived from a mouse was prepared. FIG. 8B illustrates the structure of the pX330-pFmC vector.

Furthermore, in this Experiment Example, fusion proteins in which the position and length of the mouse Cdt1-derived peptide linked to the C-terminal of the SpCas9 protein were variously modified were prepared. Specifically, a vector (SEQ ID NO: 31, hereinafter may be referred to as “pX330-pCmC”) expressing a fusion protein (hereinafter may be referred to as “Cas9-pCmC”) having no nuclear localization signal derived from the SV40 virus and in which the C-terminal of the SpCas9 protein was linked to a peptide composed of the 81st to 132nd of the Cdt1 protein derived from a mouse was prepared. FIG. 8C illustrates the structure of the pX330-pCmC vector.

In addition, a vector (SEQ ID NO: 32, hereinafter may be referred to as “pX330-pMmC”) expressing a fusion protein (hereinafter may be referred to as “Cas9-pMmC”) having no nuclear localization signal derived from the SV40 virus and in which the C-terminal of the SpCas9 protein was linked to a peptide composed of the 55th to 106th of the Cdt1 protein derived from a mouse was prepared. FIG. 8D illustrates the structure of the pX330-pMmC vector.

In addition, a vector (SEQ ID NO: 33, hereinafter may be referred to as “pX330-pNmC”) expressing a fusion protein (hereinafter may be referred to as “Cas9-pNmC”) having no nuclear localization signal derived from the SV40 virus and in which the C-terminal of the SpCas9 protein was linked to a peptide composed of the 29th to 80th of the Cdt1 protein derived from a mouse was prepared. FIG. 8E illustrates the structure of the pX330-pNmC vector.

In addition, a vector (SEQ ID NO: 34, hereinafter may be referred to as “pX330-pNNmC”) expressing a fusion protein (hereinafter may be referred to as “Cas9-pNNmC”) having no nuclear localization signal derived from the SV40 virus and in which the C-terminal of the SpCas9 protein was linked to a peptide composed of the 29th to 54th of the Cdt1 protein derived from a mouse was prepared. FIG. 8F illustrates the structure of the pX330-pNNmC vector.

In addition, a vector (SEQ ID NO: 35, hereinafter may be referred to as “pX330-pNCmC”) expressing a fusion protein (hereinafter may be referred to as “Cas9-pNCmC”) having no nuclear localization signal derived from the SV40 virus and in which the C-terminal of the SpCas9 protein was linked to a peptide composed of the 54th to 80th of the Cdt1 protein derived from a mouse was prepared. FIG. 8G illustrates the structure of the pX330-pNCmC vector.

### Experiment Example 9

### Study 1 of Subcellular Localization of Cas9 Fusion Protein

HEK293T cells, which are cells derived from human fetal kidney, were used to study subcellular localization of the fusion proteins expressed by the introduction of the expression vectors prepared in Experiment Example 8.

First, the pX330-pFmC vector, the pX330-pCmC vector, the pX330-pMmC vector, the pX330-pNmC vector, the pX330-pNNmC vector, and the pX330-pNCmC vector were introduced to the HEK293T cells, respectively. HEK293T cells with the pX330 vector introduced were also prepared for comparison.

The fusion proteins encoded by these vectors are each linked to a 3×FLAG tag peptide. Thus, each one of the Cas9 proteins can be detected by immunostaining using an anti-FLAG antibody.

Each cell was then formaldehyde-fixed and immunostained with an anti-FLAG antibody. Then, the nuclei were stained with DAPI. Each stained cell was then observed using a fluorescence microscope.

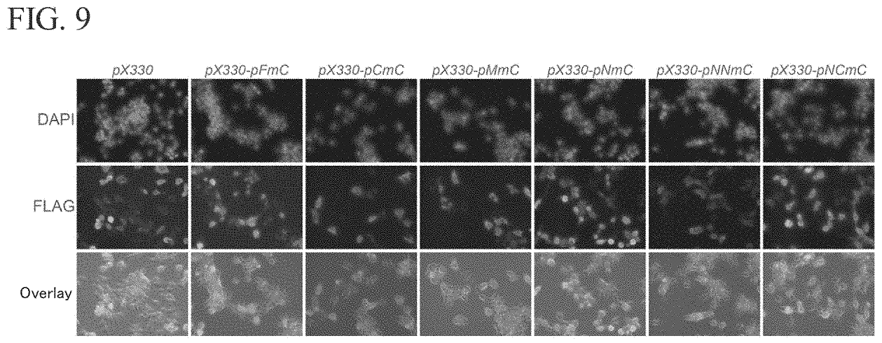

FIG. 9 shows fluorescence micrographs presenting the results of immunostaining. In FIG. 9, “pX330” represents the results of the introduction of the pX330 vector, “pX330-pFmC” represents the results of the introduction of the pX330-pFmC vector, “pX330-pCmC” represents the results of the introduction of the pX330-pCmC vector. “pX330-pMmC” represents the results of the introduction of the pX330-pMmC vector, “pX330-pNmC” represents the results of the introduction of the pX330-pNmC vector, “pX330-pNNmC” represents the results of the introduction of the pX330-pNNmC vector, and “pX330-pNCmC” represents the results of the introduction of the pX330-pNCmC vector. Also, “DAPI” represents the results of staining the nuclei with DAPI, “FLAG” represents the results of detecting each one of the Cas9 proteins by immunostaining using an anti-FLAG antibody, and “Overlay” represents the results of synthesizing the staining results using DAPI and the staining results using the anti-FLAG antibody.

As a result, it was found that the Cas9-pFmC fusion protein and the Cas9-pNmC fusion protein were localized in the nucleus. In contrast, the SpCas9 protein, the Cas9-pCmC fusion protein, the Cas9-pMmC fusion protein, the Cas9-pNNmC fusion protein and the Cas9-pNCmC fusion protein expressed by introducing the pX330 vector did not show nuclear localization.

### Experiment Example 10

### Study 2 of Subcellular Localization of Cas9 Fusion Protein

Soluble nuclear protein fractions and cytoplasmic protein fractions were separately extracted from the HEK293T cells in which each one of the fusion proteins was expressed in Experiment Example 9 using a commercially available kit (Code No. “295-73901”, Wako Pure Chemical Industries. Ltd.), and each one of the Cas9 fusion proteins was then detected using Western blotting.

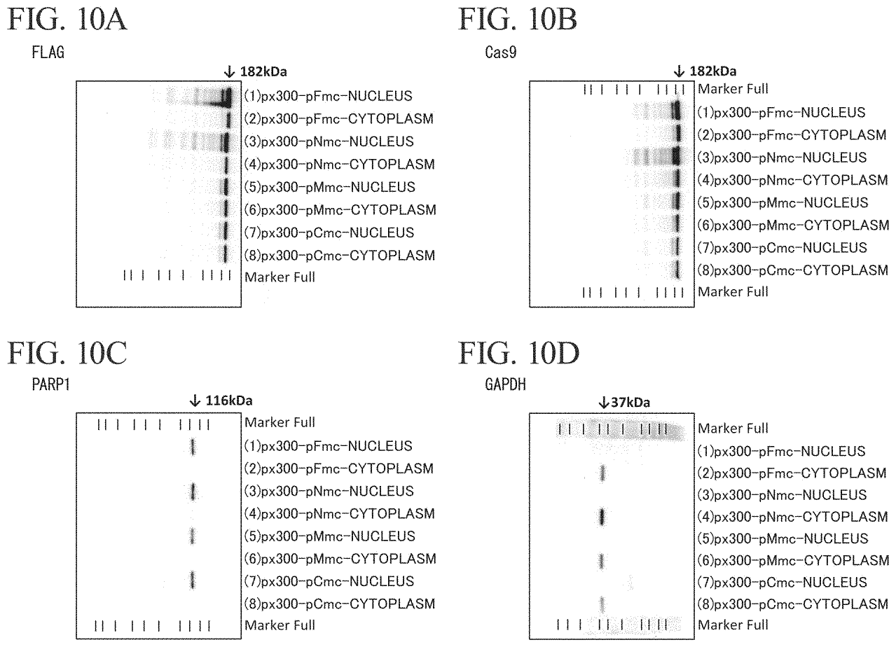

FIG. 10A is a photograph presenting the results of detecting each one of the Cas9 fusion proteins using an anti-FLAG antibody. In addition, FIG. 10B is a photograph showing the results of detecting each one of the Cas9 fusion proteins using an anti-Cas9 antibody. As a result, similar to the result of Experiment Example 9, it was confirmed that more of the Cas9-pFmC fusion protein and the Cas9-pNmC fusion protein were detected in the nucleus rather than the cytoplasm and that the Cas9-pFmC fusion protein and the Cas9-pNmC fusion protein were localized in the nucleus.

FIG. 10C is a photograph presenting the results of detecting the nuclear protein PARP1, as a control, using an anti-PARP1 antibody. FIG. 10D is a photograph showing the results of detecting the cytoplasmic protein GAPDH, as a control, using an anti-GAPDH antibody. As a result, it was confirmed that the protein fractions and cytoplasmic fractions were able to be extracted.

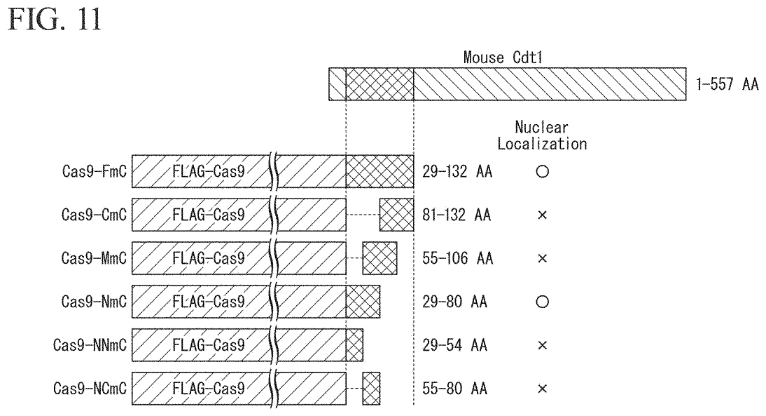

FIG. 11 is a chart summarizing the structure and nuclear localization of each one of the Cas9 fusion proteins based on the results of Experiment Examples 9 and 10. In FIG. 11. “0” indicates high nuclear localization, while “x” indicates that no nuclear localization was observed.

### Experiment Example 11

### Study of Degradability of Cas9 Fusion Protein

HEK293T cells, which are cells derived from human fetal kidney, were used to study degradability of the Cas9-pFmC fusion protein and the Cas9-pNmC fusion protein prepared in Experiment Example 8.

First, the pX330-pFmC vector and the pX330-pNmC vector were introduced into HEK293T cells, respectively. HEK293T cells with the pX330 vector introduced were also prepared for comparison.

Each cell was then formaldehyde-fixed and immunostained with an anti-FLAG antibody. Then, the nuclei were stained with DAPI. Each stained cell was then observed using a fluorescence microscope.

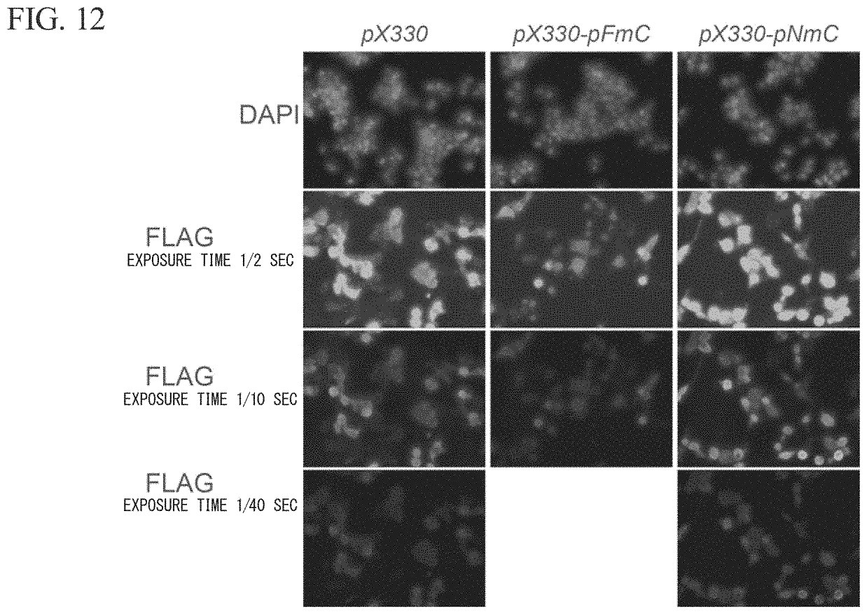

FIG. 12 shows fluorescence micrographs presenting the results of immunostaining. In FIG. 9. “pX330” represents the results of the introduction of the pX330 vector. “pX330-pFmC” represents the results of the introduction of the pX330-pFmC vector, and “pX330-pNmC” represents the results of the introduction of the pX330-pNmC vector. Also, “DAPI” represents the results of staining the nuclei with DAPI, while “FLAG” represents the results of detecting each one of the Cas9 proteins by immunostaining using an anti-FLAG antibody.

As a result, the Cas9-pNmC fusion protein was detected with higher fluorescence intensity of the anti-FLAG antibody than the Cas9-pFmC fusion protein. This result indicates that the Cas9-pNmC fusion protein is less likely to be degraded than the Cas9-pFmC fusion protein.

### INDUSTRIAL APPLICABILITY

According to an embodiment of the present invention, a modified Cas9 that improves KO efficiency and KI efficiency can be provided.

### SEQUENCE LISTING

1. A fusion protein comprising a Cas9 protein and a modifying peptide that modifies the Cas9 protein, wherein the modifying peptide includes: a peptide consists of the amino acid sequence set forth in SEQ ID NO: 29; or a peptide consists of an amino acid sequence in which one or several amino acids are deleted, substituted, or added to the amino acid sequence set forth in SEQ ID NO: 29 and having activity to localize the Cas9 protein in the nucleus by forming a fusion protein with the Cas9 protein.

2. The fusion protein according to claim 1, wherein the modifying peptide comprises: a peptide consists of the amino acid sequence set forth in SEQ ID NO: 1; or a peptide consists of an amino acid sequence in which one or several amino acids are deleted, substituted, or added to the amino acid sequence set forth in SEQ ID NO: 1 and having activity to localize the Cas9 protein in the nucleus by forming a fusion protein with the Cas9 protein.

3. The fusion protein according to claim 1, wherein the modifying peptide further comprises: a peptide consists of the amino acid sequence set forth in SEQ ID NO: 2; or a peptide consists of an amino acid sequence in which one or several amino acids are deleted, substituted, or added to the amino acid sequence set forth in SEQ ID NO: 2 and having activity to degrade the Cas9 protein in the G₁ phase by forming a fusion protein with the Cas9 protein.

4. The fusion protein according to claim 1, wherein the modifying peptide is provided at the N-terminal, at the C-terminal, or at a site that is neither the N-terminal nor the C-terminal of the Cas9 protein.

5. A nucleic acid encoding the fusion protein according to claim 1.

6. A method for producing a cell in which the genomic DNA are edited, the method comprising contacting the fusion protein according to claim 1 with the genomic DNA in a cell.

7. The method according to claim 6, wherein the cell is a fertilized egg.

8. A method of producing an animal in which the genomic DNA are edited, the method comprising contacting the fusion protein according to claim 1 with genomic DNA in a fertilized egg to obtain a fertilized egg in which the genomic DNA are edited, and growing the fertilized egg in which the genomic DNA are edited into an individual to obtain the animal in which the genomic DNA are edited.
